(19) United States
(12) Patent Application Publication
(10) Pub. No.: US 2004/0143321 A1
(43) Pub. Date: Jul. 22, 2004

(54) Expandable medical device and method for treating chronic total occlusions with local delivery of an angiogenic factor

(75) Inventors: Frank Litvack, Los Angeles, CA (US); John F. Shanley, Redwood City, CA (US); Theodore L. Parker, Danville, CA (US)

Correspondence Address: BURNS DOANE SWECKER & MATHIS L L P, POST OFFICE BOX 1404, ALEXANDRIA, VA, 22313-1404, US

(73) Assignee: Conor Medsystems, Inc.

(21) Appl. No.: 10/705,110

(22) Filed: Nov. 10, 2003

Related U.S. Application Data
(60) Provisional application No. 60/424,896, filed on Nov. 8, 2002.

(51) Int. Cl. A61M 31/00; A61F 2/06

(52) U.S. Cl. 623/1.42

(57) ABSTRACT

A method for treating blood vessel occlusions in the heart delivers an angiogenic agent from an implantable device locally to the walls of the blood vessel over an extended administration period sufficient to establish self sustaining blood vessels. An expandable medical device for delivery of angiogenic agents includes openings in the expandable medical device struts to deliver one or more angiogenic agents to promote angiogenesis. The device can sequentially deliver a plurality of agents to promote angiogenesis to treat, for example, disorders and conditions associated with chronic total occlusions.

### CROSS REFERENCE TO RELATED APPLICATIONS

This application claims priority to U.S. Provisional Application Serial No. 60/424,896, filed Nov. 8, 2002, which is incorporated herein by reference in its entirety.

### BACKGROUND OF THE INVENTION

1. Field of the Invention

The invention relates to the use of expandable medical devices to treat chronic total occlusions by delivering one or more angiogenic compositions to the wall of an artery to promote angiogenesis. The invention is also useful for the sequential delivery of a multiplicity of agents to promote angiogenesis.

### REFERENCES

Bräsen, J. H., Kivelä, A., Röser, K., Rissanen, T. T., Niemi, M., Luft, F. C., Donath, K., and Ylä-Herttuala, S. (2001) Angiogenesis, vascular endothelial growth factor and platelet-derived growth factor-BB expression, iron deposition, and oxidation-specific epitopes in stented human coronary arteries. Arterioscler. Thromb. Vasc. Biol. 21:1720-26.

Browder, T., Folkman, J., and Pirie-Shepherd, S. (2000) The hemostatic system as a regulator of angiogenesis. J. Biol. Chem. 275:1521-24.

Bukrinsky, M. I., Haggerty, S., Dempsey, M. P., Sharova, N., Adzhubel, A., Spitz, L., Lewis, P., Goldfarb, D., Emerman, M. and Stevenson, M. (1993) Nature 365:666-69.

Carmeliet, P. and Collen, D. (1999) Role of vascular endothelial growth factors and vascular endothelial growth factor receptors in vascular development in Vascular Growth Factors and Angiogenesis. Lena Claesson-Welsh, Ed. Springer-Verlag, Berlin, Heidelberg. pp. 133-158.

Davda, J. and Labhasetwar, V. (2001) An update on angiogenesis therapy. Crit. Rev. Eukaryot. Gene Expr. 11:1-21.

Isner, J. M. (2002) Myocardial gene therapy. Nature 415:234-39.

Freedman, S. B. and Isner, J. M. (2001) Therapeutic angiogenesis for ischemic cardiovascular disease. J Mol Cell Cardiol. 33:379-93.

Freedman, S. B. and Isner, J. M. (2002) Therapeutic angiogenesis for coronary artery disease. Ann. Intern. Med. 136:54-71.

Lewis, P. F. and Emerman, M. (1994) J. Virol. 68:510-6.

Morishita, R., Aoki, M., Kaneda, Y., and Ogihara, T. (2001) Gene therapy in vascular medicine: recent advances and future perspectives. Pharmacol. Ther. 91:105-14.

Naldini, L., Blomer, U., Gallay, P., Ory, D., Mulligan, R., Gage, F. H., Verma, I. M. and Trono, D. (1996) Science 272:263-67.

Nugent, M. A. and lozzo, R. V. (2000) Fibroblast growth factor-2. Int. J. Biochem. Cell Biol. 32:115-20.

Rosenfeld, M. A., Siegfried, W., Yoshimura, K., Yoneyama, K., Fukayama, M., Stier, L. E., Pääkkö, P. K., Gilardi, P., Stratford-Perricaudet, L. D., Perricaudet, M. et al. (1991) Science 252:431-34.

Simons, M. (2001) Therapeutic coronary angiogenesis: a fronte praecipitium a tergo lupi? Am. J. Physiol. Heart Circ. Physiol. 280:H1923-27.

Todd, S., Anderson, C-G., Jolly, D. J., and Craik, C. S. (2000) HIV protease as a target for retrovirus vector-mediated gene therapy. Biochim. Biophys. Acta. 1477:168-88.

Verma, I. M. and Somia, N. (1997) Nature 389:239-42.

Webster, K. A. (2000) Therapeutic angiogenesis: a case for targeted, regulated gene delivery. Crit. Rev. Eukaryot. Gene Expr. 10:113-25.

Yang, Y., Li, Q., Ertl, H. C. and Wilson, J. M. (1995) J. Virol. 69:2004-15.

Yeh, P. and Perricaudet, M. (1997) Faseb J. 11:615-23.

Zimmerman, M. A., Selzman, C. H., Raeburn, C. D., Calkins, C. M., Barsness, K., and Harken, A. H. (2001) Clinical applications of cardiovascular angiogenesis. J. Card. Surg. 16:490-97.

2. Summary of the Related Art

Chronically occluded or narrowed blood vessels prevent adequate blood flow to tissue. The treatment of chronically occluded arteries remains problematic even after a quarter century of percutaneous angioplasty. The principal limitation of conventional angioplasty for the treatment of this disorder is that a small channel through the occlusion must be created to allow for passage of a guidewire and the angioplasty device. Conventional angioplasty may be successful in approximately 50% of patients by forcing a guidewire through the occlusion, dilating with a balloon and often placing stents across the freshly opened occlusion. Restenosis or reocclusion is higher in treated chronically occluded vessels compared to treating non-occluded or narrowed vessels. Many occlusions, however, cannot be treated using this technique. A variety of alternative technologies have been developed and evaluated including but not limited to laser, atherectomy, ultrasound, spectroscopy, and thrombolysis. None of these methods have proved advantageous.

Certain forms of narrowed blood vessels are not amenable to successful surgical or percutaneous treatment. These include but are not limited to diffusely diseased blood vessels, small diameter blood vessels, tortuous blood vessels, calcified blood vessels, and vessels that supply tissue beds with impeded vascular outflow.

A number of investigations have been reported using angiogenic factors injected into or applied to the exterior of arteries. Such angiogenic factors have included proteins, DNA, or gene fragments. Preliminary results have been encouraging but not definitive. A principal limitation of prior investigations has been the inability to delivery the angiogenic factors locally and over a sustained period of time. As such, efficacy has been compromised by the suboptimal delivery of angiogenic factors.

Overview Of Angiogenesis

Blood vessel formation is an intricate process involving sequential interactions between the extra-cellular matrix (ECM), soluble and insoluble polypeptides, and cell surface receptors. The process begins during embryogenesis, as mesodermal cells differentiate into haemangioblasts that aggregate to form blood islands. The inner and outer island cells further differentiate into haematopoietic precursor cells and primitive endothelial cells (angioblasts), respectively. Basic fibroblast growth factor (bFGF) and the (VEGF-A) receptor are associated with these differentiation events (Carmeliet, P. and Collen, D. (1999)).

In a process known as vasculogenesis, the angioblasts, migrate and assemble into primitive blood capillaries (the capillary plexus) that comprise distinct luminal and exterior surfaces. Vasculogenesis involves such polypeptide factors as, VEGF-A, bFGF, fibronectin, αvβ3 integrin, VE cadherin, and transforming growth factor (TFG)-β1. The process also involves a regulatory tension between two VEGF-A receptors: VEGF receptor-2, which upregulates vasculogenesis, and VEGF receptor-1, which inhibits the process. The α5 integrin receptor may also play a role. The ECM and surrounding pericytes may infiltrate the primordial capillaries formed during vasculogenesis, causing invagination and bifurcation, resulting in capillary loops. The process is also mediated by VEGF-A, in concert with angiopoietins, the TIE receptors, and ECM polypeptides (Carmeliet, P. and Collen, D. (1999)).

In response to angiogenic factors, such as VEGF-A, the emerging capillary network gives rise to additional branches, extensions, and connections in a process called angiogenesis. During angiogenesis, the ECM of existing capillaries is proteolytically degraded by matrix metalloproteinases, as well as tPA, and uPA, at the site of the future blood vessel. Epithelial cells at the site of the ECM disruption divide and migrate toward the angiogenic factors, forming chords of endothelial cells that become new blood vessels. These emerging chords fuse with other capillaries in a process involving fibronectin and α4 integrin. VE-cadherin, Ang1, Ang2, tissue factor, TGF-β1 platelet-derived growth factor (PDGF)-B, TIE2, as well as other vascular endothelial growth factors (VEGFs), hepatocyte growth factor (HGF), insulin-like growth factor, epidermal growth factor, platelet-derived endothelial cell growth factor (PD-ECGF), platelet factor 4 (PF4), hypoxia-induced factor (HIF-1), thrombospondin (TSP-1), tumor necrosis factor (TNF), angiogenin, fibroblast growth factor receptor (FGFR), proliferin, plasminogen activator inhibitor type 1 (PAI-1), inteleukin 8 (IL-8), high molecular weigh kininogen (HMWK), and sphingosine 1-phosphate other have all been implicated in angiogenesis. Elastins and fibrillins are later deposited in the lumen of these vessels, most likely after the establishment of blood flow (Carmeliet, P. and Collen, D. (1999); Freedman, S. B. and Isner, J. M. (2002); Simons, M. (2001); Davda, J. and Labhasetwar, V. (2001); Zimmerman, M. A. et al. (2001); and references within).

The process of angiogenesis is by no means limited to embryogenesis. Angiogenesis is a natural response to hypoxia and ischemia and is intimately associated with normal physiological processes such as wound repair and placental growth. Angiogenesis is also associated with pathological diseases and conditions, including tumor growth (Freedman, S. B. and Isner, J. M. (2001); Davda, J. and Labhasetwar, V. (2001); Browder, T. et al. (2000); and references within).

In view of the importance of angiogenesis in human disease and wound repair, extensive research has been conducted to identify angiogenic agents useful for promoting angiogenesis in a clinical setting. Several angiogenic polypeptides shown to induce angiogenesis in vivo are described in greater detail, below.

Vascular Endothelial Growth Factor (VEGF):

VEGFs are a family of structurally related glycoproteins that promote proliferation and migration of endothelial cells and are expressed by epithelial tissues, neutrophils, and mononuclear cells. VEGFs also increase vascular permeability resulting in the release of a variety of plasma components. Although VEGFs share structural homology they differ with respect to heparin-binding activity. At present, the VEGF family includes VEGF (VEGF-A), VEGF-1, VEGF-2 (VEGF-C), VEGF-3 (VEGF-B), VEGF-D, VEGF-E, and another polypeptide designated placental growth factor. In addition, alternative splicing results in other isoforms of VEGF-1, i.e., VEGF121, VEGF 145, VEGF165, VEGF189, and VEGF206, wherein the subscript number refer to the number of amino acid residues in the mature polypeptide (Freedman, S. B. and Isner, J. M. (2002); Simons, M. (2001); Davda, J. and Labhasetwar, V. (2001); Zimmerman, M. A. et al. (2001); and references within).

Acidic and Basic Fibroblast Growth Factors:

Acidic FGF (aFGF, FGF-1) and basic FGF (bFGF, FGF-2) are members of a large family of polypeptides that use cell-surface heparin and heparin sulfate to mediate binding to target tyrosine kinase receptors. FGFs are ligands for various cell types and potent mitogens for endothelial cells. In response to FGF binding, endothelial cells produce proteases, such as plasminogen activator and metalloproteinases, which are involved in degredation of the extracellular matrix (Freedman, S. B. and Isner, J. M. (2002); Davda, J. and Labhasetwar, V. (2001); Nugent, M. A. and lozzo, R. V. (2000); and references within).

Hypoxia-Induced Factor (HIF-1):

HIF-1 is a transcription factor that activates several genes associated with angiogenesis, including VEGFs, VEGF receptors, and Ang-2. Under normal physiological conditions, the alpha subunit of the polypeptide is rapidly degraded; however, hypoxic conditions result in decreased degradation of the alpha subunit and increased HIF-1 activity. In addition to binding hypoxia response elements of certain angiogenesis-associated genes, HIF-1 may also stabilize RNAs by binding to the 3′ (and possibly 5′) untranslated regions, and may also be involved in cap-independent translation of angiogenesis-associated mRNAs (Simons, M. (2001); Freedman, S. B. and Isner, J. M. (2001); and references within).

Hepatocyte Growth Factor (HGF):

HGF promotes endothelial cell proliferation, migration, and invasion; VEGF production from smooth muscle cells; and protease production (Davda, J. and Labhasetwar, V. (2001); Webster, K. A. (2000); and references within).

Experimental data further suggest that multiple angiogenic factors, administered at specific times during angiogenesis, are required to mediate the formation of mature and stable blood vessels. For example, VEGF stimulates the production of thin-walled, sinusoidal vessels that lack secondary branching and complexity. However, subsequent administration of Ang1 induces further branching and recruits smooth muscle cells (and perhaps other periendothelial support cells) to the walls of the immature VEGF-induced vessels.

The identification of polypeptides involved in angiogenesis is an important step in the development of clinical therapies for patients suffering from ischemia or hypoxia. However, simple systemic treatment with angiogenic factors is likely to cause hypotension and edema (e.g., as observed with VEGF) as well as systemic toxicity, thrombocytopenia, and anemia (e.g., as observed with FGF) (Freedman, S. B. and Isner, J. M. (2001); Davda, J. and Labhasetwar, V. (2001)). Treatment of local ischemia, for example, ischemia resulting from chronic total occlusions of cardiac and peripheral arteries, requires the delivery of angiogenic agents only to selected physiological targets (see, e.g., Simons, M. (2001)). However, the absence in the art of a suitable beneficial agent delivery vehicle has frustrated attempts to deliver angiogenic factors in a clinical setting.

Expandable Medical Devices for the Delivery of Beneficial Agents

Permanent and biodegradable devices have been developed for implantation within a body passageway to maintain patency of the passageway. These devices have typically been introduced percutaneously, and transported transluminally until positioned at a desired location. These devices are then expanded either mechanically, such as by the expansion of a mandrel or balloon positioned inside the device, or expand themselves by releasing stored energy upon actuation within the body. Once expanded within the lumen, these devices, called stents, become encapsulated within the body tissue and remain a permanent implant.

Known stent designs include monofilament wire coil stents (U.S. Pat. No. 4,969,458); welded metal cages (U.S. Pat. No. 4,733,665 and U.S. Pat. No. 4,776,337); and thin-walled metal cylinders with axial slots formed around the circumference (U.S. Pat. No. 4,733,665; 4,739,762; and U.S. Pat. No. 4,776,337). Known construction materials for use in stents include polymers, organic fabrics, and biocompatible metals, such as, stainless steel, gold, silver, tantalum, titanium, cobalt based alloys, and shape memory alloys such as Nitinol.

U.S. Pat. No. 4,733,665; 4,739,762; and U.S. Pat. No. 4,776,337 disclose expandable and deformable interluminal vascular grafts in the form of thin-walled tubular members with axial slots allowing the members to be expanded radially outwardly into contact with a body passageway. After insertion, the tubular members are mechanically expanded beyond their elastic limit and thus permanently fixed within the body.

Coated stents, designed to release various beneficial agents, have shown promising results in reducing restenosis, a condition commonly associated with stent implantation. For example, U.S. Pat. No. 5,716,981 discloses a stent that is surface-coated with a composition comprising a polymer carrier and Paclitaxel (a well-known tubulin assembly inhibitor that is commonly used in the treatment of cancerous tumors).

However, a major technological obstacle facing the use of stents for the delivery of angiogenic agents is the thickness of the stent coating. Stent coatings are necessarily very thin, typically 5 to 8 microns. Since the surface area of the stent is comparatively large, the entire volume of the beneficial agent has a very short diffusion path to discharge into the surrounding tissue. This issue is especially problematic for therapies that require the prolonged delivery of a beneficial agent. While increasing the thickness of the surface coating improves drug release kinetics, it also results in an undesirable increase in overall stent thickness.

Thus, it would be desirable to provide a drug delivery stent capable of extended delivery of an angiogenic composition.

### SUMMARY OF THE INVENTION

The instant invention satisfies a need in the art by providing, an expandable medical device and method to treat total chronic occlusions by delivering one or more angiogenic agents to an implantation site to stimulate angiogenesis.

In accordance with one aspect of the present invention, a method for treating an obstructed blood vessel includes identifying an obstructed blood vessel and identifying an implantation site at or near the obstruction in the blood vessel; delivering an expandable medical device into the obstructed blood vessel to the selected implantation site; implanting the medical device at the implantation site; and delivering an angiogenic composition from the expandable medical device to tissue at the implantation site over a sustained time period sufficient to reestablish adequate blood flow to the tissue.

In accordance with another aspect of the invention, a method of delivering an angiogenic composition to an obstructed blood vessel includes:

a) identifying an obstructed blood vessel and identifying an implantation site at or near the obstruction in the blood vessel;

b) providing an expandable medical device with an angiogenic composition;

c) delivering the expandable medical device with the angiogenic composition to the implantation site; and

d) stimulating angiogenesis by sustained delivery of the angiogenic composition over a time period sufficient to create self-sustaining blood vessels.

In accordance with a further aspect of the invention, a method of delivering a series of angiogenic compositions to a chronic total arterial occlusion includes:

a) identifying an obstructed blood vessel and identifying an implantation site at or near the obstruction in the blood vessel;

b) providing an expandable medical device with a first angiogenic composition and a second angiogenic arranged for sequential delivery from the stent;

c) delivering the expandable medical device with the first and second angiogenic compositions to the implantation site; and

d) delivering the first and second angiogenic compositions sequentially at the implantation site.

In accordance with an additional aspect of the present invention, a beneficial agent delivery device includes an expandable medical device having a plurality of struts with a plurality of openings and an angiogenic composition contained in the plurality of openings in a bioresorbable matrix. The angiogenic agent and matrix are configured for administration of the angiogenic agent to a mural side of the device over a period of at least one week.

In accordance with another aspect of the invention, a beneficial agent delivery device includes an expandable medical device having a plurality of struts with a plurality of openings, a first angiogenic agent contained in the plurality of openings, and a second angiogenic agent contained in the plurality of openings. The first and second angiogenic agents are arranged in the openings for sequential delivery to tissue surrounding the device.

### BRIEF DESCRIPTION OF THE DRAWINGS

The invention will now be described in greater detail with reference to the preferred embodiments illustrated in the accompanying drawings, in which like elements bear like reference numerals, and wherein:

FIG. 1 is a cross-sectional perspective view of a portion of an expandable medical device with beneficial agent implanted in the lumen of an artery;

FIG. 2 is a perspective view of an expandable medical device showing a plurality of openings;

FIG. 3 is an expanded side view of a portion of the expandable medical device of FIG. 2;

FIG. 4 is an enlarged cross-section of an opening illustrating one or more beneficial agents provided in a plurality of layers;

FIG. 5 is an enlarged cross-section of an opening illustrating a plurality of beneficial agents provided for sequential delivery; and

FIG. 6 is an enlarged cross-section of an opening illustrating one or more beneficial agents provided in layer(s) that extend beyond a surface of the expandable medical device.

### DETAILED DESCRIPTION OF THE PREFERRED EMBODIMENTS

Definitions

As used herein, the following terms have the following meanings:

Adventitia: The outermost connective tissue layer of a blood vessel.

Angiogenic agents: Angiogenic polypeptides, angiogenic polynucleotides, angiogenic polypeptide-encoding gene therapy delivery vectors, angiogenic small molecules, or active or inactive combinations thereof.

Angiogenic compositions: Compositions comprising angiogenic agents.

Angiogenic factors: Angiogenic polypeptides.

Arteriosclerosis: Hardening of the arteries produced by degenerative or hyperplasic changes to the intima of arteries or a progressive increase in muscle and elastic tissue in arterial walls.

Atherosclerosis: The most common form of arteriosclerosis characterized by deposits of lipid material in the intima of medium and large diameter arteries, resulting in partial or total occlusion of an affected vessel.

Beneficial agent: As used herein, the term “beneficial agent” is intended to have its broadest possible interpretation and is used to include any therapeutic agent or drug, as well as inactive agents such as barrier layers, carrier layers, therapeutic layers or protective layers. Beneficial agents include but are not limited to angiogenic polypeptides, polynucleotides, and small molecules.

Beneficial layers: Biodegradable layers comprising beneficial compositions.

Biodegradable: See Bioerodible, below.

Bioerodible: The characteristic of being bioresorbable and/or able to be broken down by either chemical or physical processes, upon interaction with a physiological environment. For example, a biodegradable or bioerodible matrix is broken chemically or physically into components that are metabolizable or excretable, over a period of time from minutes to years, preferably less than one year, while maintaining any requisite structural integrity in that same time period.

Chronic total occlusion: The complete blockage of a blood vessel for an indefinite period of time causing chronic hypoxia in the tissues normally supplied by the occluded blood vessels.

Erosion: The process by which components of a medium or matrix are bioresorbed and/or degraded and/or broken down by chemical or physical processes. For example in reference to biodegradable polymer matrices, erosion can occur by cleavage or hydrolysis of the polymer chains, thereby increasing the solubility of the matrix and availability of beneficial agents, or by physical dissolution and excretion.

Erosion rate: A measure of the amount of time it takes for the erosion process to occur, usually reported in unit-area per unit-time.

Hypoxia: Condition characterized by an abnormally low oxygen concentration in affected tissues.

Intima: The innermost layer of a blood vessel.

Ischemia: Local anemia resulting from obstructed blood flow to an affected tissue.

Matrix or biocompatible matrix: The terms “matrix” or “biocompatible matrix” are used interchangeably to refer to a medium or material that, upon implantation in a subject, does not elicit a detrimental response sufficient to result in the rejection of the matrix. The matrix typically does not provide any therapeutic responses itself, though the matrix may contain or surround a beneficial agent, as defined herein. A matrix is also a medium that may simply provide support, structural integrity or structural barriers. The matrix may be polymeric, non-polymeric, hydrophobic, hydrophilic, lipophilic, amphiphilic, and the like. The matrix may be bioerodible or non-bioerodible.

Media: The middle layer of a blood vessel.

Paclitaxel: An anticancer drug that prevents depolymerization of microtubules thereby allowing initial microtubule formation but preventing subsequent rearrangement necessary for cell growth.

Pharmaceutically acceptable: The characteristic of being non-toxic to a host or patient and suitable for maintaining the stability of a beneficial agent and allowing the delivery of the beneficial agent to target cells or tissue.

a. Polymer: The term “polymer” refers to molecules formed from the chemical union of two or more repeating units, called monomers. Accordingly, included within the term “polymer” may be, for example, dimers, trimers and oligomers. The polymer may be synthetic, naturally-occurring or semisynthetic. In preferred form, the term “polymer” refers to molecules which typically have a M_{w} greater than about 3000 and preferably greater than about 10,000 and a M_{w} that is less than about 10 million, preferably less than about a million and more preferably less than about 200,000. Examples of polymers include but are not limited to, poly-α-hydroxy acid esters such as, polylactic acid (PLLA or DLPLA), polyglycolic acid, polylactic-co-glycolic acid (PLGA), polylactic acid-co-caprolactone; poly (block-ethylene oxide-block-lactide-co-glycolide) polymers (PEO-block-PLGA and PEO-block-PLGA-block-PEO); polyethylene glycol and polyethylene oxide, poly (block-ethylene oxide-block-propylene oxide-block-ethylene oxide); polyvinyl pyrrolidone; polyorthoesters; polysaccharides and polysaccharide derivatives such as polyhyaluronic acid, poly (glucose), polyalginic acid, chitin, chitosan, chitosan derivatives, cellulose, methyl cellulose, hydroxyethylcellulose, hydroxypropylcellulose, carboxymethylcellulose, cyclodextrins and substituted cyclodextrins, such as beta-cyclo dextrin sulfo butyl ethers; polypeptides, and proteins such as polylysine, polyglutamic acid, albumin; polyanhydrides; polyhydroxy alkonoates such as polyhydroxy valerate, polyhydroxy butyrate, and the like.

b. Radially inner or radially interior surface: With respect to expandable medical device struts, a radially inner or interior surface refers to a surface that has a substantially equivalent radius to that of the interior strut surface.

Radially intermediate surface: With respect to expandable medical device struts, a radially intermediate surface refers to a surface that has a substantially equivalent radius intermediate between that of the interior and exterior strut surfaces.

Restenosis: The recurrence of stenosis after a surgical procedure, including the infiltration of smooth muscle cells into the bore of an expandable medical device implanted to correct a previous chronic occlusion.

Self-sustaining blood vessels: Blood vessels that continue to perfuse tissue for a period of at least 12 months following their induction, for example, by angiogenic agents.

Sequential delivery: Delivery of beneficial agents in a specified sequence, for example where about 75% of a first agent is delivered before about 50% of a second agent is delivered.

Stenosis: A restriction or occlusion of any vessel or orifice.

Thrombosis: The formation of a thrombus (clot) within a blood vessel, often leading to partial or total occlusion of the blood vessel, leading to a condition of hypoxia in tissues supplied by the occluded blood vessel.

The present invention relates to the use of expandable medical devices, and more particularly to the use of expandable medical devices having a plurality of beneficial agent containing openings to deliver beneficial agents to an implantation site over an extended period of time. The invention also relates to the use of expandable medical devices to deliver different beneficial agents, or combinations of agents, to a wall of a blood vessel to stimulate local angiogenesis. In one embodiment of the invention, beneficial agents are delivered to one or more sites of chronic total occlusion. Disorders and conditions associated with chronic total occlusions include but are not limited to distal embolization, arterial ruptures, acute myocardial infarction, myocardial infarction, groin hematomas, contrast-induced nephropathies, angina pectoris, digital microcirculation, chronic thromboembolic pulmonary hypertension, chronic subcritical ischemia, death, and other disorders or conditions resulting from chronic total chronic occlusion of coronary arteries.

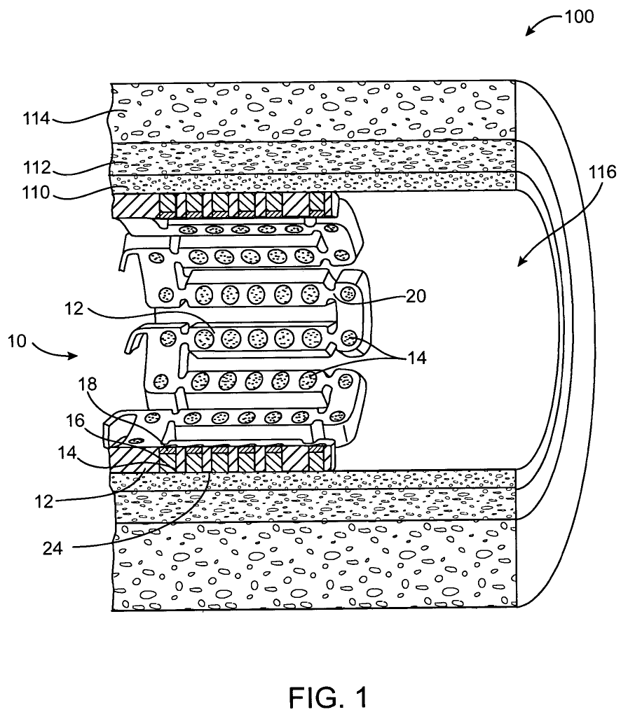

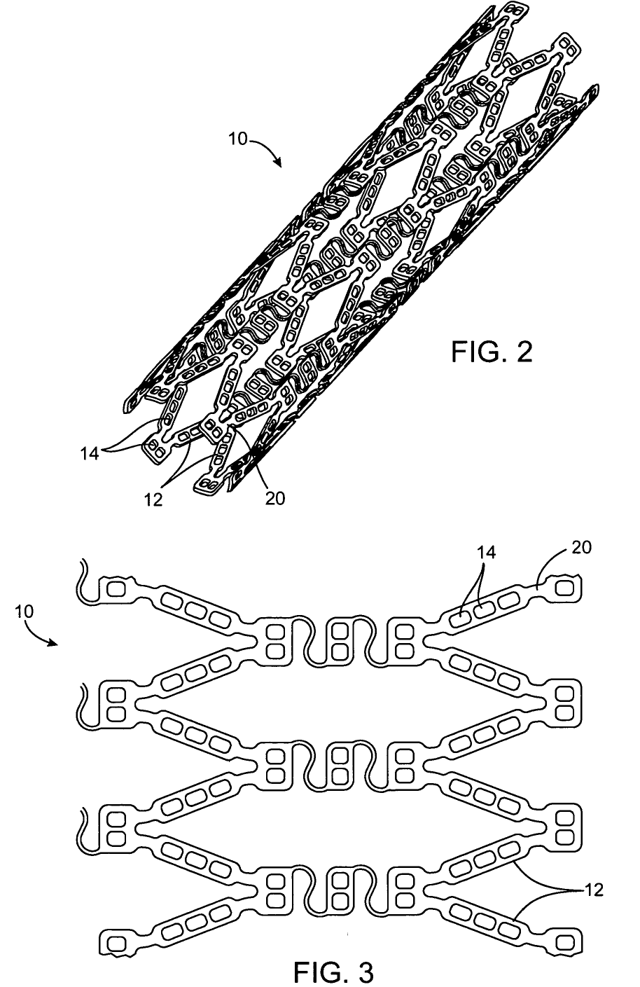

One embodiment of the expandable medical device used in the present invention, shown disposed longitudinally in an artery, is depicted in FIG. 1. Another embodiment of an expandable medical device is shown in FIGS. 2 and 3. The expandable medical devices 10, as shown in FIGS. 1-3, include a plurality of struts 12 which are interconnected by ductile hinges 40, such that as the device expands, the ductile hinges deform while the struts remain undeformed. Openings 14 in the struts 12 provide reservoirs for delivering a beneficial agent to tissue. The openings 14 in the embodiments of FIGS. 1-3 are provided in non-deforming elements of the expandable medical device. However, other device structures may also be used.

The angiogenic agents 16 are disposed in the openings 14 and may comprise one or more angiogenic polypeptides. The angiogenic polypeptides may be native or recombinant polypeptides. Examples of angiogenic polypeptides include VEGF, FGF, and HGF, and Ang1. Angiogenic polypeptides may be provided using polynucleotides encoding angiogenic polypeptides. Polynucleotides may be delivered using a gene delivery vector, including but not limited to a retrovirus vector or an adenovirus vector. The angiogenic compositions may also comprise angiogenic small molecules. Angiogenic compositions may comprise combinations of angiogenic polypeptides, polynucleotides, and small molecules. Angiogenic compositions and combinations thereof may be delivered over a period of one or two weeks or months, preferably at least one month, following expandable medical device implantation to stimulate local angiogenesis. The vessels or network of vessels created by the sustained delivery of the angiogenic composition are self-sustaining and provide blood flow to tissues, which were rendered ischemic due to a chronic total occlusion.

FIG. 1 is a cross-sectional perspective view of a portion of an expandable medical device 10 implanted in a lumen 116 of an artery 100. A wall of the artery 100 includes three distinct tissue layers, the intima 110, the media 112, and the adventitia 114. The expandable medical device 10 is similar to the expandable medical device described in U.S. Pat. No. 6,241,762, herein incorporated by reference in its entirety. U.S. Pat. No. 6,241,762 describes an expandable medical device design that remedies performance deficiencies of previous expandable medical devices by the use of ductile hinges and non-deforming stents.

FIG. 1 further depicts the peripheral struts 12 of the expandable medical device 10 having openings 14. The presence of openings 14 in the expandable medical device struts 12 containing a beneficial agent 16 provide a number of important advantages. For example, the openings 14 allow the use of a substantially larger volume of beneficial agent 16 than can be used in the case of a coating, increasing the total amount of beneficial agent available for delivery to the site of a chronic total occlusion. The ability to dispose a beneficial agent 16 in the expandable medical device 10 openings 14 also facilitates the gradual release of the beneficial agent over an extended delivery period, compared to the use of a simple coating. Furthermore, the use of openings 14 that are essentially sealed at one end by, for example, a barrier layer 18, allows the release of beneficial agents 16 in only one direction relative to the implanted expandable medical device 10. For example, as shown in FIG. 1, beneficial agents 16 may be delivered to an exterior surface 24 of the expandable medical device 10 adjacent to the intima 110 of the artery 100 while essentially no beneficial agent is directed to the lumen 116 of the artery in which the expandable medical device is implanted. The barrier layer 18 in the expandable medical device 10 openings 14 minimizes diffusion of beneficial agents 16 in the direction of the barrier layer allowing directional delivery of the agents.

FIG. 2 is a perspective view of one embodiment of an expandable medical device 10 showing a plurality of openings 14 in the struts 12 of the device. FIG. 3 is an expanded side view of a portion of the expandable medical device 10 of FIG. 2, further showing the arrangement of openings 14 in the struts 12 of the device.

In the embodiment of FIGS. 2 and 3, the struts 12 are non-deforming struts connected by ductile hinges 20. The ductile hinges 20 allow expansion or compression of the expandable medical device 10 while allowing the struts 12, and thus the openings 14 to remain undeformed during expansion or compression.

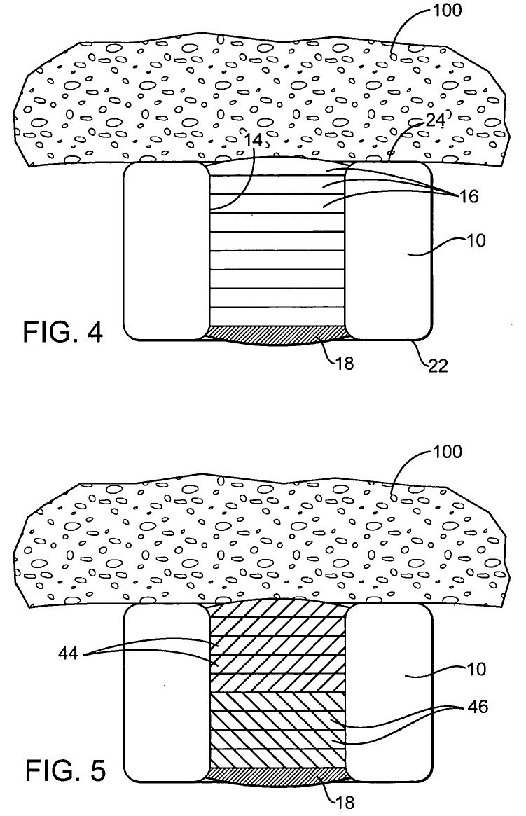

Enlarged cross-sections of openings, illustrating one or more beneficial agents provided in a plurality of layers, are shown in FIGS. 4-6. As shown in the embodiment of FIG. 4, the opening 14 in the strut 12 is provided with a plurality of layers of the beneficial agent 16 combined with a bioerodible matrix material. In one embodiment of the invention, the total depth of the opening 14 is about 50 to about 140 microns (μM) and a typical layer thickness is about 2 to about 50 microns, preferably about 12 microns. Each layer is thus individually about twice as thick as the typical coating applied to surface-coated expandable medical devices. There can be two layers in each opening 14 or as many as six to twenty layers in an opening, with a total beneficial agent thickness about 25 to about 28 times greater than a typical surface coating. According to one embodiment of the invention, the openings 14 each have a cross-sectional area of at least about 5×10⁻⁶ square inches, and preferably at least about 10×10⁻⁶ square inches.

Since each layer of beneficial agent may be created independently, individual chemical compositions and pharmacokinetic properties can be imparted to each layer. Numerous useful arrangements of layers can be formed, some of which will be described below. Each of the layers may include one or more agents 16 in the same or different proportions from layer to layer. The layers may be solid, porous, or filled with other drugs or excipients.

Although multiple discrete layers are shown for ease of illustration, the layers may be discrete layers with independent compositions or blended to form a continuous polymer matrix and agent inlay. For example, the layers can be deposited separately in layers of a drug, polymer, solvent composition which are then blended together in the openings by the action of the solvent. The agent may be distributed within an inlay uniformly or in a concentration gradient. Examples of some methods of creating such layers and arrangements of layers are described in U.S. Patent Publication No. 2002/0082680, published on Jun. 27, 2002, which is incorporated herein by reference in its entirety. The use of drugs in combination with polymers within the openings 14 allows the medical device 10 to be designed with drug release kinetics tailored to the specific drug delivery profile desired.

FIG. 4 shows an expandable medical device 10 with a simple arrangement of layers in the opening 14. The layers include identical layers of at least one beneficial agent suspended or dissolved in a bioerodible matrix that together establish a uniform, homogeneous distribution of beneficial agent. The erosion of the bioerodible matrix results in the release of beneficial agent at a release rate over time corresponding to the erosion rate of the matrix. Use of bioerodible carriers in combination with openings is especially useful, to assure essentially 100% discharge of the beneficial agent within a predetermined period of time.

The concentration of the same angiogenic agents in the layers could be varied from layer to layer, facilitating release profiles of a predetermined shape. Progressively, increasing concentrations of angiogenic agent at layers of greater depth results in the release of the agent at an approximately linear rate over time or an approximately zero order delivery profile.

Alternatively, different layers could comprise different angiogenic agents or an angiogenic agent and another therapeutic agent, providing the ability to release different agents at different times following implantation of the expandable medical device 10. In one embodiment of the invention, the different layers are eroded sequentially such that the majority of the beneficial agent in a first layer at an outer surface of the device 10 is delivered before the majority of beneficial agent of the second or underlying layer, and so forth.

FIG. 5 illustrates an alternative embodiment of an expandable medical device 10 including two beneficial agents for sequential delivery to a mural side of the device at an implantation site. In FIG. 5, a plurality of first layers 44 are provided for delivering a first beneficial agent and a plurality of second layers 46 are provided for delivering a second beneficial agent. The first and second beneficial agents are delivered in a sequential manner such that a majority of the first beneficial agent is delivered before a majority of the second beneficial agent. As in the embodiment of FIG. 4, the embodiment of FIG. 5 includes a barrier layer 18 for directing the first and second beneficial agents to the wall of the artery in which the expandable medical device is implanted.

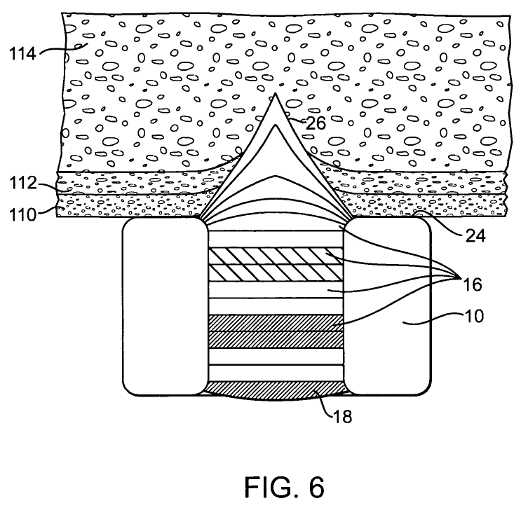

The erosion rates of individual layers may be further controlled by creating contours on the surfaces of selected layers, such as those illustrated in FIG. 6. In another example, ribs on the surface of a layer increase the overall surface area and increase the rate on initial release. Elevated or protruding portions of one layer, e.g., that extend into depressed areas in another layer, cause leading or trailing characteristics of the release profiles of the beneficial agents in the protruding or depressed layers.

Barrier layers 18 as shown in FIGS. 4-6, can be used to control beneficial agent release kinetics in several ways. First, a barrier layer 18 with a substantially non-biodegradable barrier material could be used to essentially prevent the diffusion of beneficial agents 16 in one direction, thereby insuring the delivery of beneficial agents to primarily one surface of the expandable medical device 10. Alternatively, biodegradable barrier layers 18 with predetermined erosion times longer than the erosion times of the biodegradable matrix used in the layers of the beneficial agents are also useful for directing beneficial agents to the exterior surface of the implanted expandable medical device 10 but will eventually erode providing a termination of a treatment at a predetermined time.

In the illustrated embodiments of FIGS. 4-6, the barrier layer 18 is disposed at the interior surface 22 or luminal side of the expandable medical device openings 14. Layers of beneficial agents (i.e., angiogenic agents) are disposed on top of the barrier layer 18, allowing the delivery of beneficial agents to the exterior surface 24 of the expandable medical device 10 but essentially preventing the delivery of beneficial agents to the interior surface 22 of the expandable medical device 14. The release rates of the beneficial agents can be controlled independently depending on the particular bioerodible matrix selected to deliver each agent. Release rates and release profiles can also be controlled by separating layers, layer thickness, and many other factors.

The presence of openings 14 or wells also allows layers of bioerodible matrix and therapeutic agent to be deposited beyond the exterior surface 24 (or interior surface 22) of the expandable medical device 10 as the matrix and therapeutic agent material disposed within the openings or wells serves as an anchor for a dome, cone, or other raised mass of matrix and therapeutic agent material outside the openings or wells.

FIG. 6 illustrates an extending cone 26 of matrix and therapeutic agent material outside of the expandable medical device 10. The cone can comprise, for example, the first of a number of angiogenic agents (or the first combination of angiogenic agents) 16 to be delivered to a target artery 100. Upon implantation of the expandable medical device 10, the cones 26 of matrix material are forced into contact with the intima 110 of the artery 100, delivering the beneficial agent 16 in a concentrated form with minimal opportunity for diffusion of the beneficial agents away from the target cells or tissue.

In addition, cones 26 of sufficient stiffness, as determined primarily by the matrix material, are able to mechanically penetrate the intima 110 or the intima and media 112 of the target artery 100 and deliver one or more beneficial agents 16 directly to the media 112 and/or the adventitia 114, where angiogenic factors are most likely to have an effect. As the outer cone 26 of material dissolves, new layers of bioerodible matrix are exposed, delivering additional beneficial agents 16 to the vessel wall 118. In one embodiment, only the outermost layer is conical in shape. In another embodiment, more than one layer is conical in shape. The penetration of the intima 110 or the intima and media 112 is of particular benefit for beneficial agents 16 which tend to pass slowly through or accumulate in these layers of tissue.

In one embodiment, the openings or wells contain one or more angiogenic agents, including but not limited to angiogenic polypeptides. As used herein, angiogenic polypeptides include polypeptides that directly or indirectly modulate angiogenesis in a human, including but not limited to the angiogenic polypeptides referred to above and below.

Polypeptides refer to full-length polypeptides, truncated polypeptides, chimeric polypeptides, variant polypeptides, polypeptide fragments, conjugated polypeptides, or synthetic polypeptides comprising naturally-occurring or synthetic amino acids. Any of the polypeptides may be glycosylated, phosphorylated, acylated, or otherwise modified. The invention includes the use of individual polypeptides, multiple polypeptides, polypeptides comprising multiple subunits, polypeptides requiring co-factors, and combinations thereof.

The polypeptides may be native or recombinant. The polypeptides may be obtained from natural sources or expressed in bacteria, yeast, or animal cells, including but not limited to mammalian cells. In a preferred embodiment of the invention, the polypeptides are human polypeptides. In another embodiment of the invention, the polypeptides are non-human primate polypeptides. In yet another embodiment of the invention, the polypeptide are mammalian polypeptides. In another embodiment of the invention, the polypeptides are truncated, chimeric, or variant polypeptides comprising one or more of the polypeptides referred to above.

The polypeptides may be active or inactive. Inactive polypeptides are useful, for example, for clinical experiments that require control expandable medical devices having one or more inactive beneficial agents and for blocking or modulating the activity of angiogenic receptors at some time coincident with or following expandable medical device implantation. The polypeptides may further include a proteolytic cleavage site, destruction sequence, or secondary binding site for one or more modulating agents to allow modulation of polypeptide activity, specificity, or stability, coincident with or following expandable medical device implantation.

In one embodiment, the openings or wells contain VEGF polypeptides in a bioerodible matrix. In a preferred embodiment, the VEGF polypeptide is VEGF-A or VEGF-145. In another embodiment, the openings or wells of the expandable medical device contain FGF polypeptides. In a preferred embodiment the polypeptide is bFGF or FGF-2. In yet another embodiment of the invention, the openings or wells of the expandable medical device contain one or more polypeptides selected from a matrix metalloproteinases, tPA, uPA, Ang, 1, Ang2, tissue factor, TGF-β1, PDGF-B, hepatocyte growth factor (HGF), insulin-like growth factor, epidermal growth factor, PD-ECGF, PF4, TSP-1, TNF, proliferin, plasminogen activator, IL-8, and HGF.

The angiogenic polypeptides may be conjugated to other molecules to, for example, modulate their stability, hydrophilicity, hydrophobicity, activity, or ability to interact with particular receptors, cells types, or tissues. In one embodiment of the invention, the polypeptides are conjugated to heparin or heparin sulfate. In another embodiment, the polypeptides are conjugated to naturally occurring or synthetic lipid molecules.

The practitioner will recognize that any polypeptide conjugate known in the art to be useful for, e.g., polypeptide stability, delivery, or modulation, may be used within the scope of the invention. Any number of different conjugates may be used in the instant invention. In addition, any subset or all the polypeptides used as part of the instant invention may be fully conjugated, partially conjugated, or conjugated with different molecules and disposed in the same layer or in different layers.

In another embodiment of the invention, the openings contain a plurality of different layers of beneficial agents, such that the dissolution of one layer exposes the next layer in series.

In one embodiment of the invention, a first layer or series of layers (i.e., the layers closest to the target cells) comprise VEGF and a second layer or layers (i.e., the adjacent layers disposed closer to the barrier layer) comprises an angiogenin. The delivery of VEGF to a site adjacent to a chronic total occlusion stimulates the production of immature, thin-walled, sinusoidal vessels. The subsequent delivery of an angiogenin, e.g., Ang1, induces further branching and recruits smooth muscle cells (and perhaps other periendothelial support cells) to the walls of the immature VEGF-A-induced vessels. In one example, VEGF is delivered over a period of about 4-8 weeks using an appropriately eroding bioerodible matrix. Dissolution of the VEGF-A-containing layer exposes the Ang1-containing layer. Ang1 is then delivered over a period of about 4-8 weeks using an appropriate bioerodible matrix.

In another embodiment of the invention, the first layer(s) comprises FGF and a second layer(s) comprises VEGF. In another embodiment of the invention, the first layer(s) comprises FGF and a second layer(s) comprises an angiogenin. In another embodiment of the invention, the first layer(s) comprises FGF, a second layer(s) comprises VEGF, and a third layer(s) comprises an angiogenin. In yet another embodiment of the invention, the first layer(s) comprises VEGF, a second layer(s) comprises FGF, and a third layer(s) comprises an angiogenin. In another embodiment of the invention, the first layer(s) comprises VEGF and FGF and a second layer(s) comprises an angiogenin.

In another embodiment of the invention, the first layer(s) comprises a protease capable of locally degrading the extracellular matrix of the blood vessel in which the expandable medical device is implanted. Examples of proteases that are useful for practicing the invention include but are not limited to matrix metalloproteases, uPA, and tPA. One or more subsequent layers comprise angiogenic polypeptides, or combinations of angiogenic polypeptides, such as those described above and below.

As an alternative to using angiogenic polypeptides or conjugated angiogenic polypeptides to promote beneficial effects, polynucleotides encoding angiogenic polypeptides are delivered using a gene therapy-based approach in combination with an expandable medical device. As used herein, polynucleotides refer to polynucleotides encoding one or more of the full-length, truncated, chimeric, variant, fragment, or other polypeptides referred to above.

Gene therapy refers to the delivery of exogenous genes to a cell or tissue, thereby causing target cells to express the exogenous gene product. Genes are typically delivered by either mechanical or vector-mediated methods. Mechanical methods include, but are not limited to, direct DNA microinjection, ballistic DNA-particle delivery, liposome-mediated transfection, and receptor-mediated gene transfer (Morgan, R. A. and Anderson, W. F. (1993) and references within). Vector-mediated delivery typically involves recombinant virus genomes, including but not limited to those of retroviruses, adenoviruses, adeno-associated viruses, herpesviruses, vaccinia viruses, picornaviruses, alphaviruses, and papovaviruses (Todd et al. (2000); and references within).

In one embodiment of the invention, a polynucleotide encoding an angiogenic polypeptide, or a portion of an angiogenic polypeptide, is cloned into a gene therapy delivery under control of a suitable promoter. In one embodiment of the invention, the vector is a retrovirus vector. In a preferred embodiment, the vector is a lentivirus vector. In a preferred embodiment, the retrovirus (e.g., lentivirus) vector infects and integrates into the genomes of target cells but does not generate infectious virus particles. Such retrovirus vectors typically require a packaging cell line to generate infectious particles. In another embodiment of the invention, the vector is an adenovirus vector.

The vectors may have a specific tropism for the target cell type, including for example, smooth muscle cells, vascular endothelial cells, or periocytes, or the vectors may be amphotropic, i.e., capable of infecting a variety of cell types. In one embodiment of the invention, the native or homologous promoter of the gene encoding the angiogenic polypeptide is used. In another embodiment of the invention, the promoter is, for example, a retrovirus long-terminal repeat (LTR) sequence, a cytomegalovirus (CMV) promoter, or a simian virus 40 (SV40) promoter. Target cell-specific promoters may also be useful for practicing the invention. In fact, one skilled in the art will recognize that many promoters can be used in the practice of the instant invention depending, for example, on the desired level of expression in the target cells, and the desired tissue-specific expression profiles.

Sufficiently purified vector may be provided in one or more biodegradable layers along with additional suitable pharmaceutical excipients, allowing the prolonged release of the vector and the continuous infection of new target cells. Cells infected with vector subsequently express the encoded polypeptides. Gene therapy vector delivery methods are useful, for example, for delivering any of the full-length, truncated, chimeric, variant, or fragment polypeptides, combinations of polypeptides, sequential combinations of polypeptides, or combinations thereof, described above and below. One skilled in the art will recognize the need to use different virus vectors or vectors with different cell tropisms when the particular virus vectors chosen to deliver beneficial agents do not permit super-infection of the same target cells with similar virus vectors encoding different beneficial polypeptides.

In another embodiment of the invention, polynucleotides encoding angiogenic polypeptides are delivered as naked DNA, liposome-associated DNA, or otherwise modified, conjugated, or encapsulated DNA encoding any of the full-length, truncated, chimeric, variant, or fragment polypeptides, combinations of polypeptides, sequential combinations of polypeptides, or combinations thereof, described above and below.

The invention also provides the use of small-molecule therapeutic agents that stimulate angiogenesis. Some of the small-molecule therapeutic agents include lipids, such as described in U.S. Pat. No. 4,888,324 and U.S. Pat. No. 5,756,453 which are incorporated herein by reference in their entirety; angiostatin fragments, such as described in U.S. Pat. No. 5,945,403 which is incorporated herein by reference in its entirety; nicotine, as described in U.S. Patent Publication No. 2002/0128294 which is incorporated herein by reference in its entirety; pyruvate compounds, such as described in U.S. Pat. No. 5,876,916 which is incorporated herein by reference in its entirety; and monobutyrin.

The delivery of angiogenic polypeptides and small molecules may be combined with mechanical and gene therapy-based gene delivery methods to deliver the same polypeptides or combinations of polypeptides by multiple methods or different polypeptides or combinations of polypeptides by multiple methods, simultaneously or sequentially. For example, VEGF-A polypeptide could be delivered in a first layer(s) and Ang1 could be delivered using a gene therapy vector in a second layer(s).

The angiogenic agents may be delivered over a period of weeks or months following expandable medical device implantation. The use of multiple beneficial layers allows the sequential release of different angiogenic agents, different combinations of angiogenic agents, different concentrations of angiogenic agents, or combinations thereof, for predetermined periods of time following expandable medical device implantation.

The present invention is also particularly well suited for the delivery of one or more additional therapeutic agents from a mural or luminal side of a stent in addition to the agent(s) delivered to the mural side of the stent for angiogenesis. Some murally delivered agents may include antineoplastics, antiangiogenics, angiogenic factors, antirestenotics, anti-thrombotics, such as heparin, antiproliferatives, such as paclitaxel and Rapamycin.

Some of the other therapeutic agents for use with the present invention which may be transmitted luminally or murally include, but are not limited to, antiproliferatives, antithrombins, immunosuppressants, antilipid agents, anti-inflammatory agents, antineoplastics, antiplatelets, angiogenic agents, anti-angiogenic agents, vitamins, antimitotics, metalloproteinase inhibitors, NO donors, estradiols, anti-sclerosing agents, and vasoactive agents, endothelial growth factors, estrogen, beta blockers, AZ blockers, hormones, statins, insulin growth factors, antioxidants, membrane stabilizing agents, calcium antagonists, retenoid, alone or in combinations with any therapeutic agent mentioned herein. Therapeutic agents also include peptides, lipoproteins, polypeptides, polynucleotides encoding polypeptides, lipids, protein-drugs, protein conjugate drugs, enzymes, oligonucleotides and their derivatives, ribozymes, other genetic material, cells, antisense, oligonucleotides, monoclonal antibodies, platelets, prions, viruses, bacteria, and eukaryotic cells such as endothelial cells, stem cells, ACE inhibitors, monocyte/macrophages or vascular smooth muscle cells to name but a few examples. The therapeutic agent may also be a pro-drug, which metabolizes into the desired drug when administered to a host. In addition, therapeutic agents may be pre-formulated as microcapsules, microspheres, microbubbles, liposomes, niosomes, emulsions, dispersions or the like before they are incorporated into the therapeutic layer. Therapeutic agents may also be radioactive isotopes or agents activated by some other form of energy such as light or ultrasonic energy, or by other circulating molecules that can be systemically administered. Therapeutic agents may perform multiple functions including modulating angiogenesis, restenosis, cell proliferation, thrombosis, platelet aggregation, clotting, and vasodilation. Anti-inflammatories include non-steroidal anti-inflammatories (NSAID), such as aryl acetic acid derivatives, e.g., Diclofenac; aryl propionic acid derivatives, e.g., Naproxen; and salicylic acid derivatives, e.g., aspirin, Diflunisal. Anti-inflammatories also include glucocoriticoids (steroids) such as dexamethasone, prednisolone, and triamcinolone. Anti-inflammatories may be used in combination with antiproliferatives to mitigate the reaction of the tissue to the antiproliferative.

Some of the agents described herein may be combined with additives which preserve their activity. For example additives including surfactants, antacids, antioxidants, and detergents may be used to minimize denaturation and aggregation of a protein drug, such as insulin. Anionic, cationic, or nonionic detergents may be used. Examples of nonionic additives include but are not limited to sugars including sorbitol, sucrose, trehalose; dextrans including dextran, carboxy methyl (CM) dextran, diethylamino ethyl (DEAE) dextran; sugar derivatives including D-glucosaminic acid, and D-glucose diethyl mercaptal; synthetic polyethers including polyethylene glycol (PEO) and polyvinyl pyrrolidone (PVP); carboxylic acids including D-lactic acid, glycolic acid, and propionic acid; detergents with affinity for hydrophobic interfaces including n-dodecyl-β-D-maltoside, n-octyl-β-D-glucoside, PEO-fatty acid esters (e.g. stearate (myrj 59) or oleate), PEO-sorbitan-fatty acid esters (e.g. Tween 80, PEO-20 sorbitan monooleate), sorbitan-fatty acid esters (e.g. SPAN 60, sorbitan monostearate), PEO-glyceryl-fatty acid esters; glyceryl fatty acid esters (e.g. glyceryl monostearate), PEO-hydrocarbon-ethers (e.g. PEO-10 oleyl ether; triton X-100; and Lubrol. Examples of ionic detergents include but are not limited to fatty acid salts including calcium stearate, magnesium stearate, and zinc stearate; phospholipids including lecithin and phosphatidyl choline; CM-PEG; cholic acid; sodium dodecyl sulfate (SDS); docusate (AOT); and taumocholic acid.

### EXAMPLES

### Example 1

In this example, a drug delivery stent substantially equivalent to the stent illustrated in FIGS. 2 and 3 having an expanded size of about 3 mm×17 mm is loaded with VEGF-145 in the following manner. The stent is positioned on a mandrel and a slow degrading layer or barrier layer is deposited into the openings in the stent. The barrier layer is high molecular weight PLGA provided on the luminal side to prevent substantial delivery of the angiogenic compositions to the luminal side of the device. The layers described herein are deposited in a dropwise manner and are delivered in liquid form by use of a suitable organic solvent, such as DMSO, NMP, or DMAc. The degradation rate of the barrier layer is selected so that the barrier layer does not degrade substantially until after the administration period. A plurality of layers of VEGF-145 and low molecular weight PLGA matrix are then deposited into the openings to form an inlay of drug for angiogenesis. The VEGF-145 and polymer matrix are combined and deposited in a manner to achieve a drug delivery profile which results in about 70% of the total drug released in about the first 2 days, about 100% released within about 30 days. A cap layer of low molecular weight PLGA, a fast degrading polymer, is deposited over the VEGF-145 layers to prevent the angiogenic agent from being released during transport, storage, and delivery of the stent to the implantation site.

### Example 2

In this example, a drug delivery stent substantially equivalent to the stent illustrated in FIGS. 2 and 3 having an expanded size of about 3 mm×17 mm is loaded with VEGF-145 and angiogenin in the following manner. The stent is positioned on a mandrel and a slow degrading layer or barrier layer is deposited into the openings in the stent. The barrier layer is high molecular weight PLGA provided on the luminal side to prevent substantial delivery of the angiogenic compositions to the luminal side of the device. The degradation rate of the barrier layer is selected so that the barrier layer does not degrade substantially until after the administration period.

A plurality of layers of angiogenin and low molecular weight PLGA matrix are then deposited into the openings to form an inlay of drug for angiogenesis. The angiogenin and polymer matrix are combined and deposited in a manner to achieve a drug delivery profile which results in administration in about 1 hour to about 5 days. A plurality of layers of VEGF-145 and low molecular weight PLGA matrix are then deposited into the openings to form an inlay of drug for angiogenesis. The VEGF-145 and polymer matrix are combined and deposited in a manner to achieve a drug delivery profile which results in administration in about 1 day to about 30 days. The arrangement of the VEGF-145 on the mural side and the angiogenin on the luminal side results in sequential delivery of the two agents.

A cap layer of low molecular weight PLGA, a fast degrading polymer, is deposited over the angiogenin layers to prevent the angiogenic agent from being released during transport, storage, and delivery of the stent to the implantation site.

While the invention has been described in detail with reference to the preferred embodiments thereof, it will be apparent to one skilled in the art that various changes and modifications can be made and equivalents employed, without departing from the present invention.

What is claimed is:

1. A method for treating an obstructed blood vessel comprising:
identifying an obstructed blood vessel and identifying an implantation site at or near the obstruction in the blood vessel;
delivering an expandable medical device into the obstructed blood vessel to the selected implantation site;
implanting the medical device at the implantation site; and
delivering an angiogenic composition from the expandable medical device to tissue at the implantation site over a sustained time period sufficient to reestablish adequate blood flow to the tissue.

2. The method of claim 1, wherein the angiogenic composition is disposed in openings in the expandable medical device.

3. The method of claim 2, wherein the expandable medical device comprises one or more strut elements having a inner surface and an outer surface, wherein said expandable medical device openings traverse the outer surface of said strut elements.

4. The method of claim 2, wherein the openings are provided with a barrier layer arranged at an inner surface of the expandable medical device strut.

5. The method of claim 4, wherein the angiogenic composition is disposed radially outward of the barrier layer.

6. The method of claim 1, wherein the angiogenic composition comprises one or more angiogenic polypeptides suspended in a bioerodible matrix.

7. The method of claim 6, wherein the angiogenic polypeptides are native polypeptides.

8. The method of claim 6, wherein the angiogenic polypeptides are recombinant polypeptides.

9. The method of claim 6, wherein the angiogenic polypeptides are selected from the group consisting of VEGF, FGF, and HGF.

10. The method of claim 9, wherein the angiogenic composition further comprises Ang1 polypeptides.

11. The method of claim 1, wherein the angiogenic composition includes a first agent and a second agent, wherein the first and second agents are arranged to be delivered sequentially.

12. The method of claim 11, wherein the first agent is VEGF and the second agent is angiogenin, and the first agent is delivered substantially before the second agent.

13. The method of claim 11, wherein the first agent is delivered over a period of at least one week.

14. The method of claim 11, wherein the second agent is delivered over a period of at least two weeks.

15. The method of claim 1, wherein the angiogenic composition is delivered over a period of at least one month.

16. The method of claim 1, wherein the angiogenic composition is disposed in openings in the expandable medical device and the angiogenic composition extends out of the openings to form protrusions extending from the device.

17. A method of delivering an angiogenic composition to an obstructed blood vessel, comprising the steps of:
a) identifying an obstructed blood vessel and identifying an implantation site at or near the obstruction in the blood vessel;
b) providing an expandable medical device with an angiogenic composition;
c) delivering the expandable medical device with the angiogenic composition to the implantation site; and
d) stimulating angiogenesis by sustained delivery of the angiogenic composition over a time period sufficient to create self-sustaining blood vessels.

18. The method of claim 17, wherein the angiogenic composition is disposed in openings in the expandable medical device.

19. The method of claim 18, wherein the expandable medical device comprises one or more strut elements having a inner surface and an outer surface, wherein said expandable medical device openings traverse the outer surface of said strut elements.

20. The method of claim 19, wherein the openings are provided with a barrier layer arranged at an inner surface of the expandable medical device strut.

21. The method of claim 20, wherein the angiogenic composition is disposed radially outward of the barrier layer.

22. The method of claim 17, wherein the angiogenic composition comprises one or more angiogenic polypeptides suspended in a bioerodible matrix.

23. The method of claim 22, wherein the angiogenic polypeptides are native polypeptides.

24. The method of claim 22, wherein the angiogenic polypeptides are recombinant polypeptides.

25. The method of claim 22, wherein the angiogenic polypeptides are selected from the group consisting of VEGF, FGF, and HGF.

26. The method of claim 22, wherein the angiogenic composition further comprises Ang1 polypeptides.

27. The method of claim 17, wherein the angiogenic composition includes a first agent and a second agent, wherein the first and second agents are arranged to be delivered sequentially.

28. The method of claim 27, wherein the first agent is VEGF and the second agent is angiogenin, and the first agent is delivered substantially before the second agent.

29. The method of claim 27, wherein the first agent is delivered over a period of at least one week.

30. The method of claim 27, wherein the second agent is delivered over a period of at least two weeks.

31. The method of claim 17, wherein the angiogenic composition is delivered over a period of at least one month.

32. A method of delivering a series of angiogenic compositions to a chronic total arterial occlusion, comprising the steps of:
a) identifying an obstructed blood vessel and identifying an implantation site at or near the obstruction in the blood vessel;
b) providing an expandable medical device with a first angiogenic composition and a second angiogenic arranged for sequential delivery from the stent;
c) delivering the expandable medical device with the first and second angiogenic compositions to the implantation site; and
d) delivering the first and second angiogenic compositions sequentially at the implantation site.

33. The method of claim 32, wherein the first and second angiogenic compositions are disposed in openings in the expandable medical device.

34. The method of claim 33, wherein the expandable medical device comprises one or more strut elements having a inner surface and an outer surface, wherein said expandable medical device openings traverse the outer surface of said strut elements.

35. The method of claim 33, wherein the openings are provided with a barrier layer arranged at an inner surface of the expandable medical device strut.

36. The method of claim 35, wherein the first and second angiogenic compositions are disposed radially outward of the barrier layer.

37. The method of claim 32, wherein the first and second angiogenic compositions are suspended in a bioerodible matrix.

38. The method of claim 32, wherein the first angiogenic composition is delivered over a period of at least one week.

39. The method of claim 32, wherein the second angiogenic composition is delivered over a period of at least two weeks.

40. A beneficial agent delivery device comprising:
a) an expandable medical device having a plurality of struts with a plurality of openings; and
b) an angiogenic composition contained in the plurality of openings in a bioresorbable matrix, the angiogenic agent and matrix configured for administration of the angiogenic agent to a mural side of the device over a period of at least one week.

41. The device of claim 40, wherein the openings are provided with a barrier layer arranged at an inner surface of the expandable medical device strut.

42. The device of claim 41, wherein the angiogenic composition is disposed radially outward of the barrier layer.

43. The device of claim 40, wherein the angiogenic composition comprises one or more angiogenic polypeptides suspended in a bioerodible matrix.

44. The device of claim 43, wherein the angiogenic polypeptides are native polypeptides.

45. The device of claim 44, wherein the angiogenic polypeptides are recombinant polypeptides.

46. The device of claim 44, wherein the angiogenic polypeptides are selected from the group consisting of VEGF, FGF, and HGF.

47. The device of claim 44, wherein the angiogenic composition further comprises Ang1 polypeptides.

48. The device of claim 40, wherein the angiogenic composition includes a first agent and a second agent, wherein the first and second agents are arranged to be delivered sequentially.

49. The device of claim 48, wherein the first agent is VEGF and the second agent is angiogenin, and the first agent is delivered substantially before the second agent.

50. The device of claim 48, wherein the first agent is configured to be delivered over a period of at least one week.

51. The device of claim 48, wherein the second agent is configured to be delivered over a period of at least two weeks.

52. The device of claim 40, wherein the angiogenic composition is configured to be delivered over a period of at least one month.

53. The device of claim 40, wherein the angiogenic composition disposed in openings in the expandable medical device extends out of the openings to form protrusions extending from the device.

60. A beneficial agent delivery device comprising:
a) an expandable medical device having a plurality of struts with a plurality of openings;
b) a first angiogenic agent contained in the plurality of openings; and
c) a second angiogenic agent contained in the plurality of openings,
wherein the first and second angiogenic agents are arranged in the openings for sequential delivery to tissue surrounding the device.

61. The device of claim 60, wherein the openings are provided with a barrier layer arranged at an inner surface of the expandable medical device strut.

62. The device of claim 61, wherein the first and second angiogenic compositions are disposed radially outward of the barrier layer.

63. The device of claim 60, wherein the first and second angiogenic compositions are suspended in a bioerodible matrix.

64. The device of claim 60, wherein the first and second angiogenic compositions are selected from the group consisting of VEGF, FGF, and HGF.

65. The device of claim 60, wherein the first angiogenic composition is configured to be delivered over a period of at least one week.

66. The device of claim 60, wherein the second angiogenic composition is configured to be delivered over a period of at least two weeks.
